(19) United States
(12) Patent Application Publication
(10) Pub. No.: US 2017/0196998 A1
(43) Pub. Date: Jul. 13, 2017

(54) NANOPARTICLE CONTRAST AGENT FOR EARLY DIAGNOSIS OF ALZHEIMER'S DISEASE BY MAGNETIC RESONANCE IMAGING (MRI)

(71) Applicant: The Chinese University of Hong Kong, Shatin (CN)

(72) Inventors: Lawrence William BAUM, Hong Kong (CN); Hee Lum Albert CHOW, Ma On Shan (CN); Kwok Kin CHENG, Tsing Yi (CN)

(21) Appl. No.: 14/993,816

(22) Filed: Jan. 12, 2016

(51) Int. Cl. A61K 49/18; A61K 49/00

(52) U.S. Cl. CPC A61K 49/1833; A61K 49/0004

(57) ABSTRACT

The disclosure herein relates to a novel nanoparticle that can cross the blood-brain barrier and form a bond with amyloid plaques and other related protein aggregates for detection by magnetic resonance imaging (MRI). The compositions, methods of making, and methods of use set forth herein also provide a non-invasive means for diagnosing Alzheimer's disease at an early stage.

### BACKGROUND OF THE INVENTION

Alzheimer's disease (AD) is an incurable and progressive neurodegenerative disorder. Current AD drugs only provide partial symptomatic relief and do not slow degeneration. However, many drug candidates are being tested and some of these may prove effective in delaying disease progression. Therefore, it is increasingly important to identify patients as early as possible in the disease course in order to initiate treatment before irreversible brain injury takes place. The disclosure herein is directed to a novel nanoparticle that can enter the brain and specifically bind to amyloid plaques or deposits and is therefore useful for their detection by magnetic resonance imaging (MRI). The compositions, techniques, and methods set forth herein provides a new and non-invasive means to diagnose Alzheimer's disease at an early stage.

Current methods for diagnosing Alzheimer's disease are inadequate for the type of early diagnosis described herein. For example, a radioactive positron emission tomography (PET) imaging reagent that binds amyloid plaques was recently approved for clinical use, but it suffers from the short-comings of high cost, limited availability, and toxic radioactivity. Another approach using MRI to diagnose Alzheimer's disease involves the agent A-beta, which, as a toxic ingredient, may limit the maximum safe dosage that can be administered to a patient and accordingly may limit the sensitivity of the method. The invention uses non-toxic and natural materials that have well established safety profiles and do not require further toxicological testing for securing regulatory approval. The manufacturing process is simpler and lower in cost, while non-specific biodistribution in organs other than the brain is reduced. Since amyloid plaques are indicative for AD, the ability to find these plaques earlier, which the present invention provides, will allow the possibility to slow the progression or even prevent the onset of AD.

Other methods for diagnosing Alzheimers are reported in U.S. Pat. No. 8,060,179. However, U.S. Pat. No. 8,060,179 was directed to the use of a super quantum interference device (SQUID) and not MRI for diagnosis. Additional methods of diagnosis are described in the following: Sigurdsson E M, Wadghiri Y Z, Mosconi L, Blind J A, Knudsen E, Asuni A, et al. A non-toxic ligand for voxel-based MRI analysis of plaques in AD transgenic mice. Neurobiol Aging 2008/6; 29(6):836-847; Wadghiri Y Z, Sigurdsson E M, Sadowski M, Elliott J I, Li Y, Scholtzova H, et al. Detection of Alzheimer's amyloid in transgenic mice using magnetic resonance microimaging. Magnetic Resonance in Medicine 2003; 50(2):293-302; and Yang J, Zaim Wadghiri Y, Minh Hoang D, Tsui W, Sun Y, Chung E, et al. Detection of amyloid plaques targeted by USPIO-Aβ1-42 in Alzheimer's disease transgenic mice using magnetic resonance microimaging Neuroimage 2011 4/15; 55(4):1600-1609.

The present invention provides solutions to challenges outlined above as well as to many others.

### BRIEF SUMMARY OF THE INVENTION

In one aspect, the present invention provides a magnetic resonance imaging (MRI) diagnostic agent composition, including: a superparamagnetic nanoparticle, a polymer, and at least one binder of an amyloid plaque; wherein the binder is attached to the surface of the nanoparticle; and wherein the polymer coats the nanoparticle having a binder attached to the surface of the nanoparticle.

In a second aspect, the present invention provides a method of labeling amyloid plaques for MRI detection, including contacting the composition of claim 1 with an amyloid plaque, thereby labeling amyloid plaques for MRI detection.

In a third aspect, the present invention provides a method of diagnosing a disease or condition in a patient, including administering a composition set forth herein to a patient; labeling an amyloid plaque in the patient with the composition; acquiring MRI images of the patient having the composition administered; analyzing the images to detect amyloid plaque in the patient; and diagnosing a disease or condition in a patient.

In a fourth aspect, the present invention provides a method of administering a composition set forth herein, including contacting the composition with the blood stream of a patient.

In a fifth aspect, the present invention provides a method of preparing a composition set forth herein, including preparing an alkaline solution, a polymer solution, and a metal-halide solution; mixing the alkaline solution, polymer solution, and metal-halide solution to form a mixture; contacting the mixture with an oxidizing agent.

### BRIEF DESCRIPTION OF THE DRAWINGS

FIG. 1 shows a transmission electron micrograph (TEM) image of PEG-supported iron oxide (mean particle size≈30 nm).

FIG. 2 shows an adsorption isotherm of curcumin and iron oxide (at room temperature).

FIG. 3 shows the experimental data fit on Langmuir isotherm model and that curcumin binds on a homogenous site and forms a monolayer on the iron oxide surface.

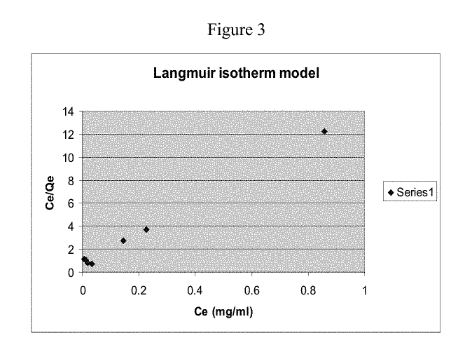

FIG. 4 shows curcumin-conjugated iron oxide nanoparticles

FIG. 5 shows confocal microscopy of fluorescent (left) and phase-contrast images (right) of amyloid plaques in consecutive APP (amyloid precursor protein) transgenic mouse brain sections. From top to bottom: Thioflavin T, curcumin, and curcumin-conjugated iron oxide.

FIG. 6 shows Prussian blue stained iron oxide particles found in APP transgenic mouse brain section 5 hours after intravenous injection of curcumin-iron oxide nanoparticles.

FIG. 7 shows bright light view of iron oxide in APP transgenic mouse brain (left). FIG. 7 also shows a fluorescent microscopy view of curcumin found on the same particle (right).

FIG. 8 shows, in the top image, a magnetic resonance image of an AD mouse brain. Black spots are iron oxide nanoparticles retained inside the brain 5 hours after injection. FIG. 8 shows, in the bottom image, a non-transgenic littermate control mouse treated and imaged under the same conditions.

FIG. 9 shows a bright view of histochemically labeled brain sections from a transgenic mouse injected with curcumin-conjugated magnetic nanoparticles.

FIG. 10 shows matches among black dots visualized by MRI, plaques labeled immunohistochemically, and plaques labeled by curcumin-conjugated magnetic nanoparticles.

FIG. 11 shows brain sections from a control mouse assayed by MRI and histochemical labeling.

FIG. 12 shows immunohistochemically labeled amyloid plaques in a transgenic mouse brain section, along with co-localization of curcumin and iron.

FIG. 13 shows the correlation between plaque density identified with immunochemistry and dark spot density identified with in vivo MRI, for transgenic mice injected with curcumin-conjugated magnetic nanoparticles.

### DETAILED DESCRIPTION OF THE INVENTION

### General

The present invention provides compositions and methods for use with MRI for diagnosing diseases involving amyloid plaques such as Alzheimer's disease, dementia, Parkinson's disease, and Lewy body disease. In part, the disclosure herein is directed to a novel nanoparticle that can cross the blood-brain barrier and bind with amyloid plaques and other similar protein aggregates such that they can be detected by magnetic resonance imaging (MRI). The compositions, techniques, and methods set forth herein also provide a non-invasive means to diagnosing Alzheimer's disease at an early stage.

### Definitions

As used herein, the term “superparamagnetic” refers to a form of magnetism, which appears, for example, in small ferromagnetic or ferrimagnetic nanoparticles. In sufficiently small nanoparticles, magnetization can randomly flip direction under the influence of temperature. The typical time between two flips is called the Neel relaxation time. In the absence of external magnetic field, when the time used to measure the magnetization of the nanoparticles is much longer than the Neel relaxation time, their magnetization appears to be in average zero. As such, they are said to be in the superparamagnetic state. In this state, an external magnetic field is able to magnetize the nanoparticles, similarly to a paramagnet. However, their magnetic susceptibility is much larger than that of paramagnets.

As used herein, the term “nanoparticle” refers to a particle having physical dimensions less than about 100 nanometers and greater than about 0.1 nanometers.

As used herein, the term “binder” refers to an agent, composition, or compound that is capable of binding or associating or complexing to an amyloid plaque.

As used herein, the phrase “amyloid plaques” refers to insoluble fibrous protein aggregates sharing specific structural traits. Plaques may also be referred to as deposits. These misfolded structures alter their proper configuration such that they erroneously interact with one another or other cell components to form insoluble fibrils. They have been associated with the pathology of more than 20 serious human diseases. Abnormal accumulation of amyloid fibrils in organs may lead to amyloidosis and may play a role in various neurodegenerative disorders.

As used herein, the phrase “protein aggregate” refers to the product of protein aggregation, which is a biological phenomenon where misfolded proteins aggregate, accumulate, and/or, clump together either intracellularly or extracellularly. These protein aggregates are often toxic and have been implicated in a wide variety of diseases known as amyloidoses, including Alzheimer's, Parkinson's and prion disease.

As used herein, the term “indicative” means to have the characteristics of a certain disease or to suggest the presence of status of a certain disease. For example, amyloid plaques are indicative of Alzheimer's disease because they are commonly associated with this disease. A patient having amyloid plaques may have Alzheimer's disease, and therefore such plaques may indicate, e.g., to a doctor, the status as to whether or not the patient has Alzheimer's disease.

As used herein, the term “hydrophilic” refers to a chemical group having a tendency to repel non-polar or uncharged chemical groups, e.g., hexane, and to attract polar or charged chemical groups, e.g., water. “Hydrophilic” also refers to a chemical that tends to dissolve in, mix with, or be wetted by water.

As used herein, the term “hydrophobic” refers to a chemical group having a tendency to attract non-polar or uncharged chemical groups, e.g., hexane, and to repel polar or charged chemical groups, e.g., water. “Hydrophobic” also refers to a chemical that tends not to dissolve in, mix with, or be wetted by water.

As used herein, the phrase “hydrodynamic particle size” refers to the smallest diameter of a hypothetical three-dimensional sphere into which a particle of the present invention could be encapsulated.

As used herein, the term “biodegradable” refers to the ability of a composition to be broken down, particularly into innocuous products by the action of living organisms.

As used herein, the term “amphiphilic” is used to describe a chemical compound as possessing both hydrophilic and lipophilic hydrophobic properties.

As used herein the term “copolymer” refers to a polymer derived from two (or more) monomeric species, as opposed to a homopolymer where only one monomer is used. For example, given monomeric species A and B, an alternating copolymer may have the form of -A-B-A-B-A-B-A-B-A-B. For example, given monomeric species A and B, a random copolymer may have the form of -A-A-B-A-B-B-A-B-A-A-A-B-B-B-B-A. For example, given monomeric species A and B, a block copolymer may have the form of -(A-A-A)-(B-B-B)-(A-A-A)-(B-B-B)-(A-A-A)-.

### Compositions

In some embodiments, the present invention provides a magnetic resonance imaging (MRI) diagnostic agent composition, which includes a superparamagnetic nanoparticle, a polymer, and at least one binder of an amyloid plaque. In some of these embodiments, the binder is attached to the surface of the nanoparticle. In some of these embodiments, the polymer coats the nanoparticle having a binder attached to the surface of the nanoparticle.

In some embodiments, the nanoparticles described herein have a first layer that includes a polymer that adheres to and coats the surface of the nanoparticle. In some embodiments, the nanoparticles described herein has a second layer that includes at least one binder of an amyloid plaque. In some embodiments, the binder of an amyloid plaque is attached to the surface of the nanoparticle. In some embodiments, the binder of an amyloid plaque is attached to the surface of the nanoparticle which is not already bonded to the polymer. In some embodiments, the binder of an amyloid plaque which is attached to the surface of the nanoparticle is capable of binding to an amyloid plaque. In certain embodiments, the nanoparticle includes a core which includes the superparamagnetic material. In some embodiments, the polymer and the binder of the amyloid plaque are adhered or bonded to the surface of the superparamagnetic nanoparticle. In some embodiments, the polymer and the binder of the amyloid plaque may be closely associated on the surface of the nanoparticle. In some embodiments, the binder of the amyloid plaque extends beyond the surface of the polymer even though both the polymer and the binder of the amyloid plaque are attached to the surface of the nanoparticle. In yet other embodiments, both the polymer and the binder of an amyloid plaque are bonded to the surface of the nanoparticle and the binder of the amyloid plaque is sufficiently exposed to be capable of binding to an amyloid plaque.

In some embodiments, the present invention provides a magnetic resonance imaging (MRI) diagnostic agent composition, including: a superparamagnetic nanoparticle, a polymer, and at least one binder of an amyloid plaque; wherein the binder is attached to the surface of the nanoparticle; and wherein the polymer coats the nanoparticle having a binder attached to the surface of the nanoparticle. In some embodiments, the polymer and the binder are both attached to the surface of the nanoparticle. In certain embodiments, the polymer and the binder are intimately associated on the surface of the nanoparticle. In other embodiments, the polymer coats both the binder and the surface of the nanoparticle. In some embodiments, the polymer coats the surface of the a nanoparticle but does not prevent the binder, which is attached to the surface of the nanoparticle, from binding to an amyloid plaque.

In some embodiments, the present invention provides that the binder is of an amyloid plaque. In other embodiments, the present invention provides that the binder is of an amyloid deposit. In certain embodiments, the amyloid plaque is selected from the group consisting of protein aggregates, A-beta aggregates, and synuclein. In some embodiments, the amyloid plaque is indicative of a disease selected from the group consisting of Alzheimer's disease, dementia, Parkinson's disease, and Lewy body disease.

In some embodiments, the present invention provides that the amyloid plaque is capable of forming a chemical bond with curcumin.

In some embodiments, the present invention provides that the nanoparticle includes iron. In certain embodiments, the present invention provides that the nanoparticle includes magnetite. In other embodiments, the present invention provides that the nanoparticle includes a metal selected from the group consisting of iron, gold, platinum, silver, and cobalt. In yet other embodiments, the present invention provides that the nanoparticle includes a semiconductor selected from the group consisting of cadmium selenide, cadmium sulfide, lead selenide, lead sulfide, zinc selenide, zinc sulfide, and combinations thereof. In some embodiments, the nanoparticle includes iron oxide.

In some embodiments, the present invention provides nanoparticles wherein the nanoparticle's dimensions are less than 30 nm. In certain embodiments, the composition's hydrodynamic particle size is less than 300 nm. In some embodiments, the hydrodynamic particle size of about 10 nm to about 300 nm.

In some embodiments, the present invention provides compositions wherein the binder is a bioflavonoid or a dye. In some of these embodiments, the binder is a dye selected from the group consisting of curcumin, Congo red, Thioflavin T/S, quercetin, epicatechin, hesperidin, rutin, and tangeritin. In certain embodiments, the dye is curcumin.

In some embodiments, the present invention provides compositions the polymer is hydrophilic. In some embodiments, the hydrophilic polymer is selected from the group consisting of polyethylene glycol (PEG), dextran, polyvinylpyrrolidone (PVP), fatty acids, polyvinyl alcohol (PVA), polyacrylic acid, polypeptides, phosphorylcholine, poly(D, L-lactide), poly(N-isopropylacrylamide) (PolyNIPAAM), chitosan, gelatin, polylactic-co-glycolic acid (PLGA), poly caprolactone (PCL), and poly(butyl)cyanoacrylate (PBCA). In some of these embodiments, the hydrophilic polymer is polyethylene glycol (PEG).

In some embodiments, the present invention provides compositions further including a biodegradable amphiphilic copolymer. In some embodiments, the biodegradable amphiphilic copolymer is selected from the group consisting of polylactic-co-glycolic acid (PLGA), polycaprolactone (PCL), polylactic acid (PLA), polyethylene glycol (PEG), methoxypolyethylene glycol (MePEG), polyethylene oxide (PEO), polybutadiene (PBD), d-α-tocopheryl polyethylene glycol 1000 succinate, PEG-PLA, PEG-PCL, PEG-PLGA, MePEG-PLA, MePEG-PCL, MePEG-PLGA, PEO-PBD, and Vitamin E TPGS.

In some embodiments, the present invention provides compositions further including a hydrophilic polymer stabilizer. In some embodiments, the hydrophilic polymer stabilizer is selected from the group consisting of polyvinyl pyrrolidone (PVP), polyvinyl alcohol (PVA), polyethylene glycols (PEG), hydroxypropyl methylcellulose (HPMC), and Poloxamer, polylactic-co-glycolic acid (PLGA), poly caprolactone (PCL), polylactic acid (PLA), poly(butyl)cyanoacrylate (PBCA), and chitosan.

In some embodiments, the present invention provides compositions further including a cationic surfactant selected from the group consisting of benzalkonium chloride, benzethonium chloride, and cetrimide.

In some embodiments, the present invention provides compositions further including an anionic surfactant selected from the group consisting of docusate sodium and sodium lauryl sulfate.

In some embodiments, the present invention provides compositions further including a non-ionic surfactant selected from the group consisting of glyceryl monooleate, sorbitan esters, polyoxyethylene sorbitan fatty acid esters, and polyoxyethylene alkyl ethers. In some embodiments, the non-ionic surfactant is a sorbitan ester selected from the group consisting of sorbitan monolaurate, sorbitan monooleate, sorbitan monopalmitate, sorbitan sesquioleate, and sorbitan trioleate. In some embodiments, the non-ionic surfactant is a polyoxyethylene sorbitan fatty acid ester selected from the group consisting of polysorbate 20, polysorbate 40, polysorbate 60, polysorbate 65, polysorbate 80, and polysorbate 85. In some other embodiments, the non-ionic surfactant is a polyoxyethylene alkyl ether selected from the group consisting of polyethylene glycol monocetyl ether, polyethylene glycol monolauryl ether, polyethylene glycol monooleyl ether, and polyethylene glycol monostearyl ether. In some embodiments, the poloxamer is selected from the group consisting of P124, P188, P237, P338, and P407.

In some embodiments, the present invention provides compositions wherein the nanoparticle is iron oxide and the dye is curcumin. In some embodiments, the present invention provides compositions having a calculated maximum loading of 75 milligrams of curcumin per gram of iron oxide. In some embodiments, the present invention provides compositions having a calculated minimum loading of 5 milligrams of curcumin per gram of iron oxide.

### Pharmaceutical Compositions

The pharmaceutical compositions of the present invention encompass compositions made by admixing a compound, nanoparticle, or composition of the present invention and a pharmaceutically acceptable carrier and/or excipient or diluent. Such compositions are suitable for pharmaceutical use in an animal or human.

The pharmaceutical compositions of the present invention include a compound, nanoparticle, or composition described herein, or a pharmaceutically acceptable salt thereof, as an active ingredient and a pharmaceutically acceptable carrier and/or excipient or diluent. A pharmaceutical composition may optionally contain other therapeutic ingredients.

The compounds of the present invention can be combined as the active ingredient in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein. The compositions may be prepared by any of the methods well-known in the art of pharmacy.

The compositions include compositions suitable for topical, parenteral, pulmonary, nasal, rectal or oral administration. The most suitable route of administration in any given case will depend in part on the nature and severity of the conditions being diagnosed. Other preferred compositions include compositions suitable for systemic (enteral or parenteral) administration. Systemic administration includes oral, rectal, sublingual, or sublabial administration. The compositions may be administered by injection, e.g., via a syringe, subcutaneously, intravenously, intramuscularly, or intraperitoneally.

Compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art.

Compositions for systemic administration include, but are not limited to, dry powder compositions consisting of the composition as set forth herein and the powder of a suitable carrier and/or excipient. The compositions for systemic administration can be represented by, but not limited to, tablets, capsules, pills, syrups, solutions, and suspensions.

In some embodiments, the present invention provides compositions further including a pharmaceutical surfactant.

In some embodiments, the present invention provides compositions further including a cryoprotectant. In some embodiments, the cryoprotectant is selected from the group consisting of glucose, sucrose, trehalose, lactose, sodium glutamate, PVP, HPβCD, CD, glycerol, maltose, mannitol, and saccharose.

In some embodiments, the present invention provides a pharmaceutical composition including the composition of claim 1 and a pharmaceutically acceptable excipient. In some of these embodiments, the pharmaceutically acceptable excipient includes a salt or a diluent.

In some embodiments, the present invention provides compositions including an effective amount of the composition of claim 1. In some embodiments, the composition is formulated for oral administration or intravenous administration and includes the composition of claim 1 and at least one member selected from the group consisting of an aqueous solution and a buffer solution.

### Method of Preparing Superparamagnetic Nanoparticles

The nanoparticles of the present invention may be prepared by the methods set forth herein. For example, curcumin conjugated iron oxide nanoparticle can be prepared by the following procedure.

In some embodiments, iron oxide nanoparticles can be prepared by the oxidation-precipitation principle, e.g., Tada M, Hatanaka S, Sanbonsugi H, Matsushita N, Abe M. Method for synthesizing ferrite nanoparticles ˜30 nm in diameter on neutral pH condition for biomedical applications, J. Appl. Phys. 2003; 93(10):7566-7568; and Konwarh R, Saikia J P, Karak N, Konwar B K. Poly(ethylene glycol)-magnetic nanoparticles-curcumin′ trio: Directed morphogenesis and synergistic free-radical scavenging. Colloids and Surfaces B: Biointerfaces 2010 12/1; 81(2):578-586. In some embodiments, a mixture of an alkaline solution, e.g., KOH (1 mol/L) with 2% polyethylene glycol (PEG), is added drop-wise into a metal halide, e.g., FeCl₂ solution until the pH reaches approximately 7.8. Under continuous stirring, hydrogen peroxide is added to yield a dark black precipitate. The products are purified, washed by de-ionized water and then acetone, and dried.

In some embodiments, iron oxide nanoparticles can be synthesized by the reverse co-precipitation method described in, for example, Aono H, Hirazawa H, Naohara T, Maehara T, Kikkawa H, Watanabe Y. Synthesis of fine magnetite powder using reverse coprecipitation method and its heating properties by applying AC magnetic field. Materials Research Bulletin 2005; 40(7): 1126-1135. In some embodiments, a base solution (e.g., NaOH dissolved in deoxygenated purified water) is added drop-wise to an iron solution (e.g., FeCl₃.6H₂O and FeSO₄.7H₂O dissolved in deoxygenated purified water at a ratio of 2 mol Fe³⁺: 1 mol Fe²⁺) while stirring. A dark black precipitate is formed during the addition of the base solution. Stirring of the mixture is maintained overnight, after which the precipitate is washed by water, re-suspended in dimethylformamide, and stored at 4° C.

The methods set forth herein are useful for preparing iron oxide nanoparticles that are approximately 10 to 30 nm in size.

The methods set forth herein are useful for preparing nanoparticles that are loaded with curcumin at a maximum loading of about 75 mg per gram of iron oxide. The maximum loading is defined herein as the loading for which curcumin forms a monolayer surface on the nanoparticle. In some embodiments, the maximum loading of curcumin is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 mg per gram of iron oxide. In other embodiments, the maximum loading of curcumin is 76, 77, 78, 79, 80, 81, 82, 83, 84, 85, 86, 87, 88, 89, 90, 91, 92, 93, 94, 95, 96, 97, 98, 99, or 100 mg per gram of iron oxide. The loading of curcumin on the nanoparticles can also be defined in terms of the mole ratio of curcumin to iron oxide. In some embodiments, nanoparticles are loaded with curcumin in a range from about 0.05:1 to about 1:1 mole ratio of curcumin to iron oxide. In some embodiments, nanoparticles are loaded with curcumin in a range from about 0.05:1 to about 0.5:1, in a range from about 0.3:1 to about 0.75:1, in a range from about 0.55:1 to about 1:1, in a range from about 0.05 to about 0.25, in range from about 0.2:1 to about 0.4:1, from about 0.35:1 to about 0.55:1, from about 0.5:1 to about 0.7:1, from about 0.65:1 to about 0.85:1, or from about 0.8:1 to about 1:1 mole ratio of curcumin to iron oxide. In some embodiments, nanoparticles loaded with curcumin at about 0.4:1 mole ratio of curcumin to iron oxide maintained their size during subsequent removal of solvent through dialysis.

In some embodiments, the nanoparticles are prepared using high power sonication. In some embodiments, the nanoparticles are dispersed in dimethylformamide under ultra-sonication for a set period of time (e.g. 1, 2, 3, 4, 5, 6, 7, 8, 9, or 10 minutes). In some embodiments, the suspension is further dried by vacuum concentrator.

In some embodiments, the nanoparticles are encapsulated in a polymeric micelle. In some embodiments, a PEG-PLA co-block polymer is dissolved in an organic phase while nanoparticles are dispersed in an aqueous phase. In other embodiments, a PEG-PLA block copolymer and the curcumin conjugated nanoparticles are both provided in an organic phase while an aqueous phase is also provided. In certain embodiments, both phases are co-injected into a multi-inlet vortex mixer. In some embodiments, the nanoparticles are subjected to high energy generation during rapid mixing. In certain embodiments, the nanoparticles are separated and prevented from aggregation. In some embodiments, polyvinyl pyrrolidone (PVP) or polyvinyl alcohol (PVA) is added into the solution. In some embodiments, the PVP or PVA are dissolved in an aqueous layer that is co-injected with the organic phase into a vortex mixer.

In some embodiments, the solvent remaining in the nanoparticle suspension is eliminated by dialysis or centrifugal filtration. In certain embodiments, drying is achieved by adding cryogenic protectant such as sucrose, mannitol, beta cyclodextrin, or glucose, and then co-freezing with the nanosuspension for freeze drying. In some embodiments, the methods include storing the dried nanoparticles. In some embodiments, after storing the nanoparticles, polysorbate 80 or mannitol is added to a solution of the nanoparticles in order to reconstitute them.

In some embodiments, the present invention provides a method of labeling amyloid plaques for MRI detection, including contacting a composition, described herein, with an amyloid plaque, thereby labeling amyloid plaques for MRI detection. In some of these embodiments, the amyloid plaque is selected from the group consisting of protein aggregates, A-beta aggregates, and synuclein. In certain embodiments, the amyloid plaque is indicative of a disease selected from the group consisting of Alzheimer's disease, dementia, Parkinson's disease, and Lewy body disease.

In some embodiments, the present invention provides a method of diagnosing a disease or condition in a patient, including administering a composition, described herein, to the patient; labeling an amyloid plaque in the patient with the composition; acquiring MRI images of the patient having the composition administered; analyzing the images to detect amyloid plaque in the patient; and thereby diagnosing a disease or condition in a patient.

In some embodiments, the present invention provides a method wherein acquiring MRI images includes operating an MRI instrument using the following MRI parameters: T2*−FLASH (2D); TE=25 ms, TR=400 ms, FA=20⁰; FOV=2×2, MTX=400×400; In-plane resolution=50 μm²; slice thickness=0.5 mm; and NEX=80, scan time=3.5 hours.

In some embodiments, the present invention provides a method of administering the composition, described herein, including contacting the composition with the blood stream of a patient.

In some embodiments, the present invention provides a method of preparing a composition described herein, including preparing an alkaline solution, a polymer solution, and a metal-halide solution; mixing the alkaline solution, polymer solution, and metal-halide solution to form a mixture; and contacting the mixture with an oxidizing agent.

In some of these embodiments, the methods include using a confined impinging jet mixer or multi-inlet vortex mixer to prepare the composition by flash nanoprecipitation.

In some embodiments, the methods include drying the composition by freeze drying, spray drying or vacuum concentration.

### Method of Diagnosing

In some embodiments, the present invention provides methods of assisting the diagnosis of a disease where amyloid plaques are present, such as Alzheimer's disease, dementia, Parkinson's disease, and Lewy body disease. In some embodiments, the methods include administering a composition of the present invention to a patient. In some embodiments, the methods further include allowing the composition of the present invention to bind or complex with an amyloid plaque. In some embodiments, the methods further include using MRI to image the amyloid plaque that is bound to a composition of the present invention. In some embodiments, the methods further include diagnosing a patient as having, or as likely to have, a disease where amyloid plaques are present, such as Alzheimer's disease, dementia, Parkinson's disease, and Lewy body disease. In some embodiments, the diagnosing involves analyzing an MM image for contrast that indicates the presence of amyloid plaques. In some embodiments, the contrast is induced in the MRI image by the composition of the present invention. In certain embodiments, the diagnosing is for an early stage of a disease recited herein.

### Patient Populations

The compositions and nanoparticles described herein are useful for diagnosing a variety of diseases. In certain embodiments, the compositions and nanoparticles that are described herein are useful for diagnosis in a person that is at least 40 years old; or that is at least 45 years old; or that is at least 50 years old; or that is at least 55 years old; or that is at least 60 years old; or that is at least 65 years old; or that is at least 70 years old; or that is at least 75 years old; or that is at least 80 years old; or that is at least 85 years old; or that is at least 90 years old; or that is at least 95 years old; or that is at least 100 years old.

Conventional diagnostic methods may be used to supplement the methods of the present invention.

### Administration

The compositions, agents, and nanoparticles described herein may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

For adult humans, dosages of the compositions of the present invention range from about 0.5 to about 2 g, in a single administration. In some embodiments for adult humans, greater or lesser dosages may be used, and multiple administrations may be used. The exact dosage will depend upon the mode of administration, the composition involved, on the diagnosis desired, form in which administered, the subject to be treated and the body weight of the subject to be treated, and the preference and experience of the physician or veterinarian in charge.

Generally, the compositions of the present invention can be dispensed in unit dosage form including preferably from about 1 to 500 mg of active ingredient together with a pharmaceutically acceptable carrier per unit dosage. For storage and use, these preparations preferably contain a preservative to prevent the growth of microorganisms.

Kits providing a unit dosage of the compositions set forth herein are contemplated as within the present invention. Kits providing many unit dosages of the compositions set forth herein are contemplated as within the present invention. Still further, kits providing several unit dosages of the compositions set forth herein are contemplated as within the present invention. In some embodiments, the kits of the present invention include a unit dosage of a pharmaceutical compositions set forth herein. In certain embodiments, the kits of the present invention include many unit dosages of a pharmaceutical compositions set forth herein. In certain other embodiments, the kits of the present invention include a unit dosage of a pharmaceutical composition set forth herein.

Administration of an appropriate amount the candidate compound may be by any means known in the art such as, for example, oral or rectal, parenteral, intraperitoneal, intravenous, subcutaneous, subdermal, intranasal, or intramuscular.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

Injection solutions and suspensions can be prepared from sterile powders, granules, and tablets of the kind previously described. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Compositions of the invention may be used in combination with other drugs that may also be useful in the treatment, prevention, suppression of a neurological or psychological disorder. Such other drugs may be administered, by a route and in an amount commonly used therefor, contemporaneously or sequentially with a compound of the invention. When a compound of the invention is used contemporaneously with one or more other drugs, a pharmaceutical composition in unit dosage form containing such other drugs and the compound is preferred. When used in combination with one or more other active ingredients, the compound of the present invention and the other active ingredients may be used in lower doses than when each is used singly.

In the pharmaceutical compositions of the present invention for oral, sublingual, subcutaneous, intramuscular, intravenous, local or rectal administration, the active principle, by itself or in association with another active principle, can be administered to animals and humans in unit forms of administration mixed with conventional pharmaceutical carriers. The appropriate unit forms of administration include oral forms such as tablets, gelatin capsules, powders, granules and solutions or suspensions to be taken orally, sublingual and buccal forms of administration, aerosols, implants, subcutaneous, intramuscular, intravenous, intranasal or intraocular forms of administration and rectal forms of administration.

### EXAMPLES

### Example 1—Synthesis and Characterization of Curcumin Conjugated Iron Oxide Nanoparticle

Iron oxide nanoparticles were fabricated by the oxidation-precipitation principle. Techniques from the following were also employed: Tada M, Hatanaka S, Sanbonsugi H, Matsushita N, Abe M. Method for synthesizing ferrite nanoparticles ˜30 nm in diameter on neutral pH condition for biomedical applications, J. Appl. Phys. 2003; 93(10):7566-7568; and Konwarh R, Saikia J P, Karak N, Konwar B K. ‘Poly(ethylene glycol)-magnetic nanoparticles-curcumin’ trio: Directed morphogenesis and synergistic free-radical scavenging. Colloids and Surfaces B: Biointerfaces 2010 12/1; 81(2):578-586.

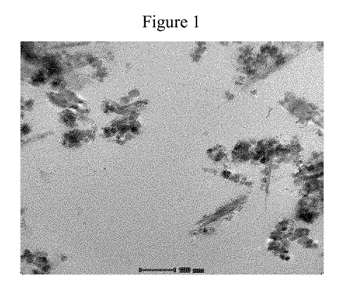

A mixture of an alkaline solution of KOH (1 mol/L) with 2% polyethylene glycol (PEG) solution was added drop-wise into a FeCl₂ solution until the pH reached 7.8. Under continuous stirring, hydrogen peroxide was added to yield a dark black precipitate. The final iron oxide products were purified, washed by de-ionized water and then acetone, and dried. The particle size of the iron oxide was observed to be in a range of 10 to 30 nm. Particles were observed to be separated by PEG. FIG. 1 shows a transmission electron microscopy (TEM) image of PEG-supported iron oxide.

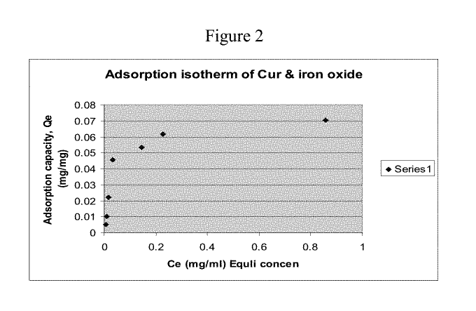

Based on the absorption isotherm study, curcumin preferentially binds the iron oxide surface, with a calculated maximum loading of 75 mg of curcumin per gram of iron oxide. FIGS. 2 & 3 show the adsorption isotherm and Langmuir model, which indicate that Curcumin is forming a monolayer and binds at a homogeneous site on the iron oxide surface.

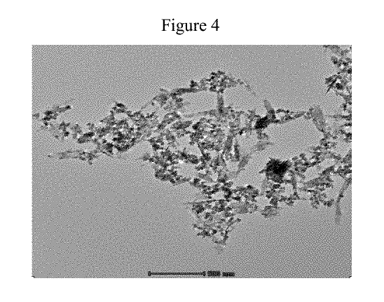

Curcumin-conjugated iron oxide was made under high power sonication using, in part, techniques from the above references and also Zhang G, Guo B, Wu H, Tang T, Zhang B T, Zheng L, He Y, Yang Z, Pan X, Chow H, To K, Li Y, Li D, Wang X, Wang Y, Lee K, Hou Z, Dong N, Li G, Leung K, Hung L, He F, Zhang L, Qin L, A delivery system targeting bone formation surfaces to facilitate RNAi-based anabolic therapy, Nat Med 2012; 18(2):307-314. The Curcumin solution was mixed with pre-dispersed iron oxide, in dimethylformamide, under ultra-sonication for 8 minutes. The suspension is further dried by vacuum concentrator. FIG. 4 shows the Curcumin-conjugated PEG-supported iron oxide nanoparticles.

### Example 2—Encapsulation of Curcumin-Conjugated Iron Oxide by Flash Nanoprecipitation (FNP) Method

Curcumin-conjugated iron oxide were encapsulated inside a polymeric micelle. PEG-PLA co-block polymer was dissolved in an organic phase while curcumin-conjugated iron oxide was dispersed in an aqueous phase. Both phases were co-injected into a multi-inlet vortex mixer. The PLA tail of PEG-PLA co-block polymer was favored to adhere to the curcumin-coated surface and form a micelle structure therewith. Due to the high energy generated during the rapid mixing, the nanoparticles are separated and prevented from aggregation. In order to enhance the storage stability, polyvinyl pyrrolidone (PVP) or polyvinyl alcohol (PVA) is added into the solution.

### Solvent Removal and Drying Process

The solvent remaining in the nanoparticle suspension was eliminated by dialysis or centrifugal filtration. Drying was achieved by adding cryogenic protectant such as sucrose, mannitol, beta cyclodextrin, or glucose, and then co-freezing with the nanosuspension for freeze drying. The dried nanoparticles can be stored indefinitely and then re-suspended in aqueous solution. In order to enhance the blood brain barrier penetration abilities of the nanoparticles, polysorbate 80 or mannitol is added to the reconstituted solution before use. Relevant techniques and materials can be found in the following: Ren T, Xu N, Cao C, Yuan W, Yu X, Chen J, et al. Preparation and Therapeutic Efficacy of Polysorbate-80-Coated Amphotericin B/PLA-b-PEG Nanoparticles. Journal of Biomaterials Science, Polymer Edition 2009; 20(10):1369-1380; and Sun W, Xie C, Wang H, Hu Y. Specific role of polysorbate 80 coating on the targeting of nanoparticles to the brain. Biomaterials 2004 7; 25(15):3065-3071.

Comparison with Existing Commercial Technologies

Recent approaches to diagnose AD by detecting amyloid β plaques include radioactive dye imaged by positron emission tomography (PET) and superparamagnetic nanoparticles imaged by magnetic resonance imaging (MRI). Some of the advantages of the present invention over existing commercially available technology, e.g. PET product (Amyvid™) by Eli Lilly, are presented in Table 1.

TABLE 1
Comparison of different amyloid imaging approaches
for diagnosis of Alzheimer's disease

| | | Iron | Iron |
|---|---|---|---|
| | | nanoparticles | nanoparticles |
| | Amyvid ™ | conjugated with | coated with |
| | (Eli Lilly) | Aβ₁₋₄₂ peptide | curcumin |
| Toxicity | Increase long | Aβ₁₋₄₂ peptide | Curcumin |
| | term cumulative | is neurotoxic. | is safe. |
| | radiation | | |
| | exposure | | |
| Manufacturing | Complicated | Complicated | Easy |
| process | | | |
| Cost | >USD3000 | >USD1000 | <USD50 |
| Stability | Short half-life | Moderate half-life | Very stable |
| Diagnostic tools | PET (available in | MRI (available in | MRI |
| | few hospitals) | many hospitals) | (available in |
| | | | many hospitals) |
| Spatial resolution | Low | High | High |
| Special training to | Yes | No | No |
| read image results | | | |
| Non-specific | High | High | Low |
| uptake (by tissue | | | |
| other than brain) | | | |

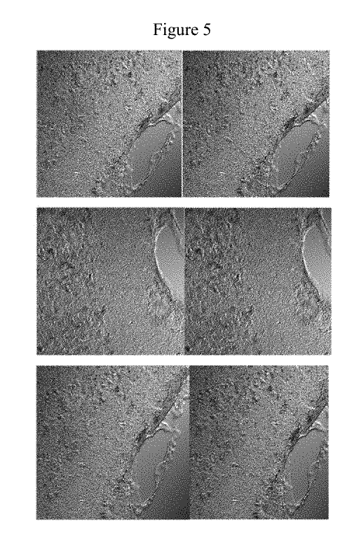

The formulation of Curcumin-conjugated magnetic nanoparticles is described herein. The size of the iron core is <30 nm, and the final hydrodynamic particle size is <300 nm. These particles have been tested in vitro and in vivo for amyloid plaque binding ability and blood-brain barrier penetration, and in vivo for MRI. FIG. 5 shows confocal fluorescent and phase-contrast images of Thioflavin T, curcumin, and curcumin-conjugated iron oxide nanoparticles applied to consecutive sections of an APP (amyloid precursor protein) transgenic mouse brain. All three reagents bind amyloid plaques.

### Example 3—Penetration of the Blood-Brain Barrier by Curcumin-Conjugated Magnetic Nanoparticle

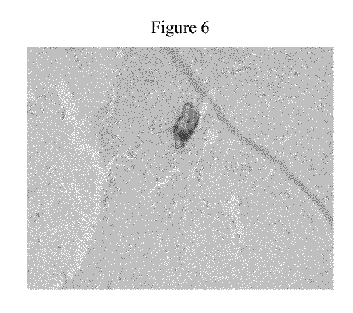

In vivo tests have been performed to evaluate the ability of the curcumin-conjugated iron oxide nanoparticles to penetrate the blood-brain barrier. The nanoparticles are injected into a vein of APP transgenic mice. Five hours after injection, mice were sacrificed and the brains harvested for histochemical staining of iron by Prussian blue reaction. The brain sections were treated with acidified ferrocyanides, and any trace of iron (Fe⁺³) turned a bright blue color. FIG. 6 shows the iron oxide particles in a mouse brain section.

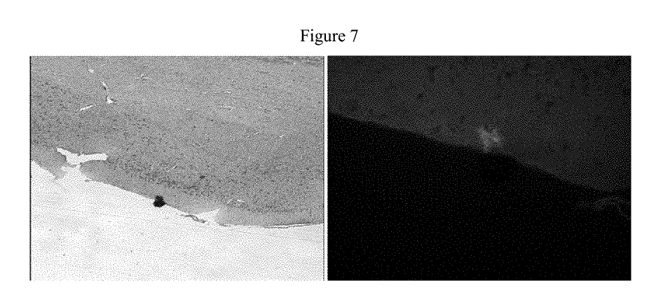

In order to confirm that Curcumin remains conjugated with iron oxide, sections in which iron oxide was found were further examined by fluorescence microscopy since curcumin exhibits fluorescence under UV light. FIG. 7 shows a section viewed under bright light and UV light. A trace amount of curcumin could be identified from the fluorescent image. These images indicate that curcumin-conjugated iron oxide can penetrate the blood-brain barrier of transgenic mice, with curcumin remaining conjugated to the iron oxide surface.

### Example 4—Magnetic Resonance Imaging and Immunochemistry of Amyloid Plaque Targeting by Curcumin-Conjugated Magnetic Nanoparticles

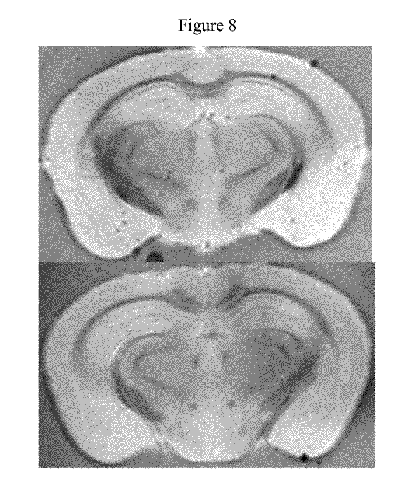

The curcumin-conjugated iron oxide nanoparticles were also tested in vivo for MR imaging and disease specificity. APP transgenic mice and non-transgenic littermate controls were given intravenous injections of the same dose of nanoparticle suspension. Their brains were harvested 5 hours after injection and fixed in an agarose gel for ex vivo MRI. FIG. 8 showed many black spots in MRI of the APP mouse brain (upper image) but not in the control brain (bottom image). Thus, iron-curcumin nanoparticles are only specifically retained and visualized by MRI in the brains of AD mice but not control mouse.

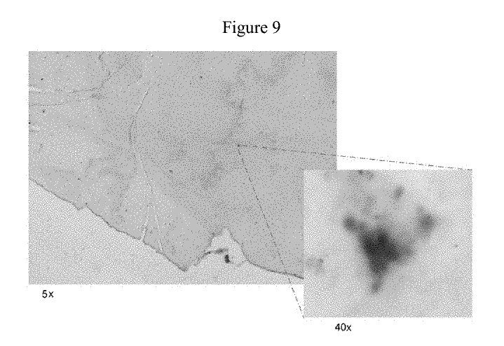

FIG. 9 shows a typical brain section of an APP transgenic mouse injected with curcumin-conjugated magnetic nanoparticles. The red spots indicate amyloid plaques stained by a mixture of 4G8 and 6E10 monoclonal antibodies to Aβ peptide, and blue indicates iron oxide. The magnified view demonstrates that iron oxide was in close proximity to an amyloid plaque.

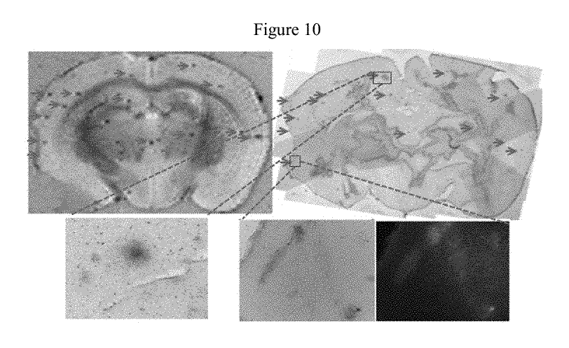

FIG. 10 shows a matched MRI (left) and double-stained APP transgenic mouse brain section (right). Many of the dark spots found in MRI co-localized with immunolabeled amyloid plaques (red) and iron oxide (blue). The inset is a 40× magnification of the selected area, showing amyloid plaques (red) and iron (blue) in the bright field image (left) and plaques (orange) in the fluorescent image (right). The fluorescence was orange because curcumin emits at 520 nm, while the fluorescent label used for immunolabeling the plaques emits at 570 nm. The combination of these yellow and red emitted signals results in a bright orange color. The images show that circumin-conjugated magnetic nanoparticles are able to bind amyloid and can be visualized by MRI or immunohistochemistry.

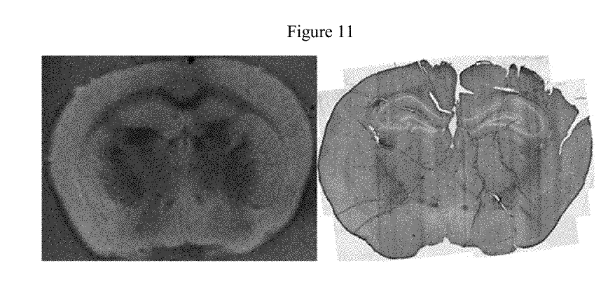

FIG. 11 shows a section of a non-transgenic control mouse of the same age after injection of curcumin-conjugated magnetic nanoparticles. No iron oxide signal was detected by MRI (left). As expected, no amyloid plaques (red) or iron (blue) was detected on the matched histology section (right).

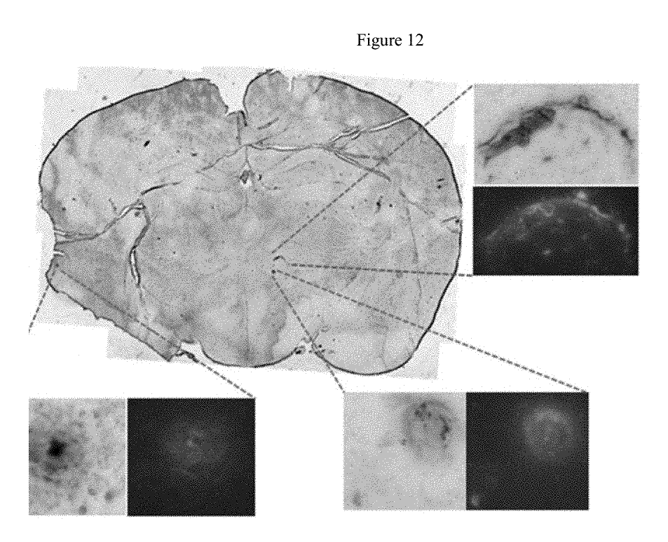

Brain sections of APP transgenic mice injected with curcumin-conjugated magnetic nanoparticles (FIG. 12) were observed for co-localization of curcumin, iron oxide and amyloid plaques. In the insets, the bright views (left) show iron oxide in blue, and the fluorescent views (right) show amyloid plaques in red and curcumin in yellow (or orange for co-localized amyloid and curcumin).

### Example 5—Curcumin-Conjugated Magnetic Nanoparticles as a Diagnostic Agent

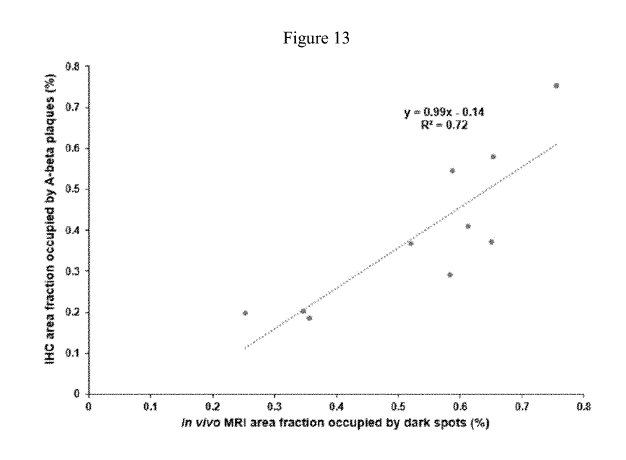

To determine whether curcumin-conjugated magnetic nanoparticles can be used as an AD diagnosis agent, it is necessary to correlate immunohistochemical and in vivo MRI results. Based on previous results, in vivo images could predict well the actual Aβ plaque locations (shown in immunohistochemistry). Ten random in vivo MRI brain sections from three transgenic mice injected with curcumin-conjugated magnetic nanoparticles were selected, and their matched immunohistochemistry sections were analyzed by a blinded operator. FIG. 13 shows the linear regression of spot density in MRI versus Aβ density in immunohistochemistry. They are strongly correlated (Pearson's correlation, p=0.002), and the R² value is 0.72. The result shows that in vivo MRI images of Tg mice injected with curcumin-conjugated magnetic nanoparticles were a good predictor of Aβ plaques found in immunohistochemistry. Curcumin-conjugated magnetic nanoparticles can thus be used clinically for diagnostic imaging of Aβ plaques.

Although the foregoing invention has been described in some detail by way of illustration and example for purposes of clarity of understanding, one of skill in the art will appreciate that certain changes and modifications may be practiced within the scope of the appended claims. In addition, each reference cited herein is incorporated by reference in its entirety to the same extent as if each reference was individually incorporated by reference. Where a conflict exists between the instant application and a reference provided herein, the instant application shall dominate.

1. A magnetic resonance imaging (MRI) diagnostic agent composition, comprising:
a superparamagnetic nanoparticle, a polymer, and a binder of an amyloid plaque;
wherein the binder is attached to the surface of the nanoparticle; and
wherein the polymer coats the nanoparticle having a binder attached to the surface of the nanoparticle.

2. The composition of claim 1, wherein the amyloid plaque is selected from the group consisting of protein aggregates, A-beta aggregates, and synuclein.

3. The composition of claim 1, wherein the amyloid plaque is indicative of a disease selected from the group consisting of Alzheimer's disease, dementia, Parkinson's disease, and Lewy body disease.

4.-35. (canceled)

36. A pharmaceutical composition comprising the composition of claim 1 and a pharmaceutically acceptable excipient.

37. The composition of claim 36, wherein the pharmaceutically acceptable excipient includes a salt or a diluent.

38. The composition of claim 36, comprising an effective amount of the composition of claim 1.

39. (canceled)

40. A method of labeling amyloid plaques for MRI detection, comprising contacting the composition of claim 1 with an amyloid plaque, thereby labeling amyloid plaques for MRI detection.

41. The method of claim 40, wherein the amyloid plaque is selected from the group consisting of protein aggregates, A-beta aggregates, and synuclein.

42. The method of claim 40, wherein the amyloid plaque is indicative of a disease selected from the group consisting of Alzheimer's disease, dementia, Parkinson's disease, and Lewy body disease.

43. A method of diagnosing a disease or condition in a patient, comprising:
administering the composition of claim 1 to the patient;
labeling an amyloid plaque in the patient with the composition;
acquiring MRI images of the patient having the composition administered;
analyzing the images to detect amyloid plaque in the patient; and
diagnosing a disease or condition in a patient.

44. The method of claim 45, wherein the acquiring MRI images comprises operating an MRI instrument using the following MRI parameters:
T2*−FLASH (2D);
TE=25 ms, TR=400 ms, FA=20⁰;
FOV=2×2, MTX=400×400;
In-plane resolution=50 μm²;
slice thickness=0.5 mm; and
NEX=80, scan time=3.5 hours.

45. The method of claim 43, wherein the disease or condition is selected from the group consisting of Alzheimer's disease, amyloid plaque disease, multiple sclerosis, Parkinson's disease, Lewy body disease, dementia, and stroke.

46.-48. (canceled)

49. A method of preparing the composition of claim 1, comprising:
preparing an alkaline solution, a polymer solution, and a metal-halide solution;
mixing the alkaline solution, polymer solution, and metal-halide solution to form a mixture;
contacting the mixture with an oxidizing agent.

50. The method of claim 1, wherein the oxidizing agent is hydrogen peroxide.

51. The method of claim 1, comprising:
using a confined impinging jet mixer or multi-inlet vortex mixer to prepare the composition by flash nanoprecipitation.

52. (canceled)

53. A magnetic resonance imaging (MRI) diagnostic agent composition, comprising:
a superparamagnetic nanoparticle core;
a first layer surrounding the core and comprising a polymer;
a second layer surrounding the core and comprising a binder.

54. The composition of claim 53, wherein the binder is a binder of an amyloid plaque.

55. The composition of claim 54, wherein the amyloid plaque is selected from the group consisting of protein aggregates, A-beta aggregates, and synuclein.

56.-71. (canceled)
